(19) United States
(12) Patent Application Publication
(10) Pub. No.: US 2008/0032311 A1
(43) Pub. Date: Feb. 7, 2008

(54) Low volume mixing of sample

(76) Inventor: Zhenghua Ji, Wilmington, DE (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/499,106

(22) Filed: Aug. 4, 2006

(51) Int. Cl. G01N 33/53; C12M 3/00

(52) U.S. Cl. 435/7.1; 435/287.2

(57) ABSTRACT

A method for low volume mixing includes suspending particles in a solution having an analyte and agitating or heating the solution. Heating the solution heats the particles suspended in the solution, thereby increasing movement of the particles. The movement of the suspended particles mixes the analyte within the solution.

### BACKGROUND

Low-volume mixing is useful in a variety of industrial and scientific pursuits. For example, low-volume mixing is important when detecting analytes within a sample. Analytes, such as genetic material, are substances within a sample that scientists desire to detect and/or measure. Low volume samples may be precious materials or rarely existing materials, such as biological substance, materialized evidence substance, and the like.

An example of applications for low-volume mixing includes detection systems for diagnosing medical conditions and mapping DNA sequences. In such systems a sample containing one or more analytes is placed onto a microarray, which may be a slide that contains an array of micro-sized spots. Each spot reacts with a particular analyte, and a scientist can detect the presence or absence of an analyte by observing whether the spot reacts (hybridizes) when exposed to the sample. Additionally, a single microarray can contain several different types of spots so that different analytes can be simultaneously detected in a single sample.

When analyzing a sample, it is important to mix analytes within the sample to produce quality hybridization and ensure that spots on the microarray are exposed to all of the analytes within the sample. This task is especially difficult given the very low volume of sample that is placed on the microarray.

### SUMMARY

In general terms, this patent relates to low-volume and localized mixing of a sample containing an analyte.

One aspect is a method for low volume mixing. The method comprises providing a fluid having suspended particles and an analyte; and heating the fluids thereby increasing movement of the suspended particles, wherein the movement of the suspended particles mixes the analyte within the fluid.

Another aspect is a system for low volume mixing. The system comprises a substrate configured to hold a fluid having an analyte and suspended particles. A heating element is configured to heat the substrate, wherein heating the substrate heats the particles suspended within the fluid causing the suspended particles to mix the analyte within the fluid.

Another aspect is a system for low volume mixing. The system comprises a container configured to retainably hold a fluid having an analyte and particles suspended in the fluid. An agitation element is configured to impart movement to the container, wherein imparting movement to the container causes the suspended particles to move within the solution, thereby mixing the analyte within the solution.

Another aspect is a kit for low volume mixing. The kit comprises a microarray and a plurality of polymer particles having a Browning motion when exposed to heat, wherein the polymer particles increase movement of an analyte suspended in a sample and across the surface of the microarray upon exposure of the polymer particles to a predetermined level of heat.

### BRIEF DESCRIPTION OF THE DRAWINGS

FIG. 1 illustrates an exemplary embodiment of an apparatus for localized mixing of a low-volume sample containing at least one analyte;

FIG. 2 illustrates one exemplary embodiment of particles suspended in the sample of the of the exemplary apparatus of FIG. 1;

FIG. 3 illustrates movement of the particles suspended in the sample of the exemplary apparatus of FIG. 1;

FIG. 4 illustrates another exemplary embodiment of an apparatus for localized mixing of a low-volume sample containing at least one analyte;

FIG. 5 illustrates movement of the particles suspended in the sample of the exemplary apparatus of FIG. 4; and

FIG. 6 illustrates an exemplary embodiment of an apparatus for localized mixing of a low-volume sample containing at least one analyte.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

Unless defined otherwise, all technical and scientific terms used herein have the same meaning as commonly understood by one of ordinary skill in the art to which this invention belongs. Still, certain elements are defined below for the sake of clarity and ease of reference.

An “array”, unless a contrary intention appears, includes any one-, two- or three-dimensional arrangement of addressable regions bearing a particular chemical moiety or moieties (for example, biopolymers such as polynucleotide sequences) associated with those regions. An array is “addressable” in that it has multiple regions of different moieties (for example, different polynucleotide sequences) such that a region (also referenced as a “feature” or “spot” of the array) at a particular predetermined location (an “address”) on the array will detect a particular target or class of targets (although a feature may incidentally detect non-targets of that feature). Note that the finite small areas on the array which can be illuminated and from which any resulting emitted light can be simultaneously (or shortly thereafter) detected, define pixels which are typically substantially smaller than a feature (typically having an area about 1/10 to 1/100 the area of a feature). Array features may be separated by intervening spaces. In the case of an array, the “target” is a moiety in a mobile phase (typically fluid), to be detected by probes (“target probes”) which are bound to the substrate at the various features. However, either of the “target” or “target probes” may be the one which is to be evaluated by the other (thus, either one could be an unknown mixture of polynucleotides to be evaluated by binding with the other). An “array layout” refers to one or more characteristics of the features, such as feature positioning on the substrate, one or more feature dimensions, and an indication of a moiety at a given location. The array “substrate” includes everything of the array unit behind the substrate front surface. “Hybridizing” and “binding”, with respect to polynucleotides, are used interchangeably.

A “biopolymer” is a polymer of one or more types of repeating units. Biopolymers are typically found in biological systems and particularly include polysaccharides (such as carbohydrates), and peptides (which term is used to include polypeptides and proteins) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

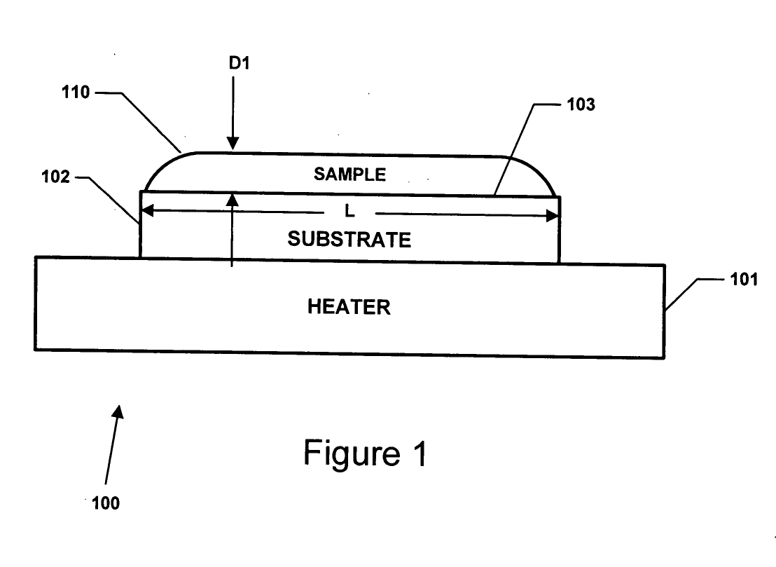

Referring to FIG. 1, an exemplary embodiment of an apparatus 100 for localized mixing of a low-volume sample containing at least one analyte includes a substrate 102 and a heater 101. The substrate 102 is configured to hold a low volume of sample 110 containing at least one analyte. The substrate 102 includes one or more target spots 103 designed to react with one or more of the analytes in the sample 110. In one example embodiment, the substrate 102 includes at least one microarray.

In some embodiments, the substrate 102 holds the sample 110 having a length L and a depth D1 via surface tension. The sample volume is measured by the product of L and D1. The low-volume in all embodiments has no specific range but is characterized by the ratio L/D1, which is much greater than 1 in one possible embodiment although other ratios are possible. In one such an embodiment, for example, L/D1 is greater than about 10. In another embodiment L/D1 is greater than about 100. In yet another embodiment L/D1 is greater than about 1000. In the exemplary embodiment, the depth D1 of the sample 110 held on the substrate 102 ranges from about 0.01 mm to about 10 mm, L is in the range from about 10 mm to about 100 mm, and the sample volume is in the range from about 1 μl to about 1000 μl, although other dimensions are possible.

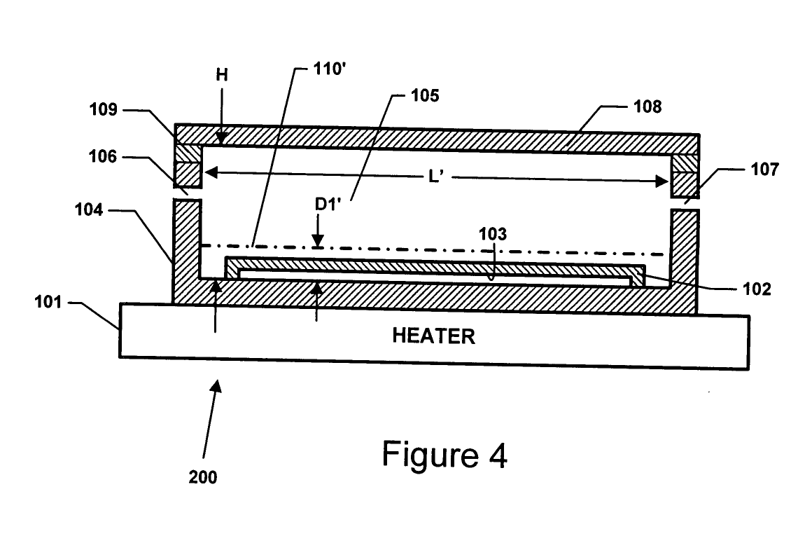

In other embodiments, shown in FIGS. 4-6, the substrate 102 is positioned within a container 104 configured to house a sample 110′, the sample 110′ having a length L′ and a depth D1′, which are sized to receive the substrate 102.

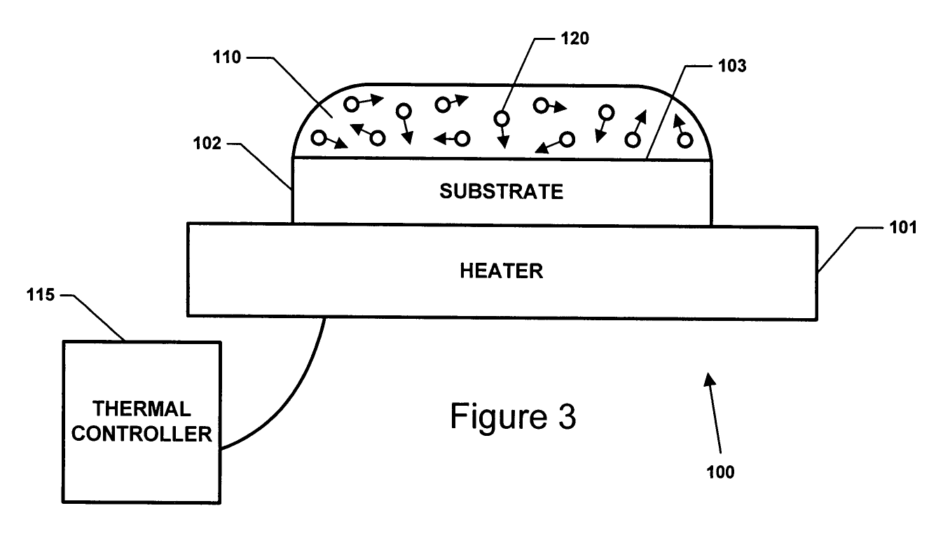

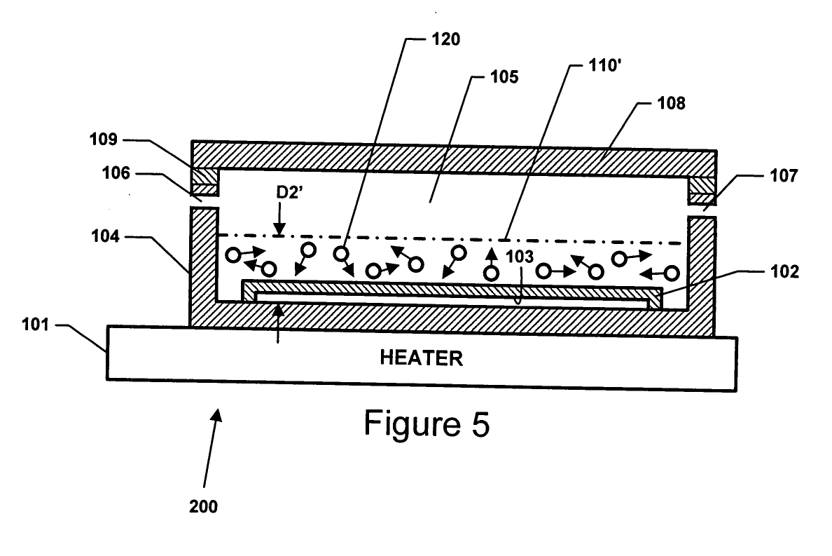

The heater 108 is configured to alter the temperature of the substrate 102, thereby altering the temperature of the sample 110. The possible configurations and functions of the heater 101 will be discussed in greater detail herein with reference to FIGS. 3 and 5.

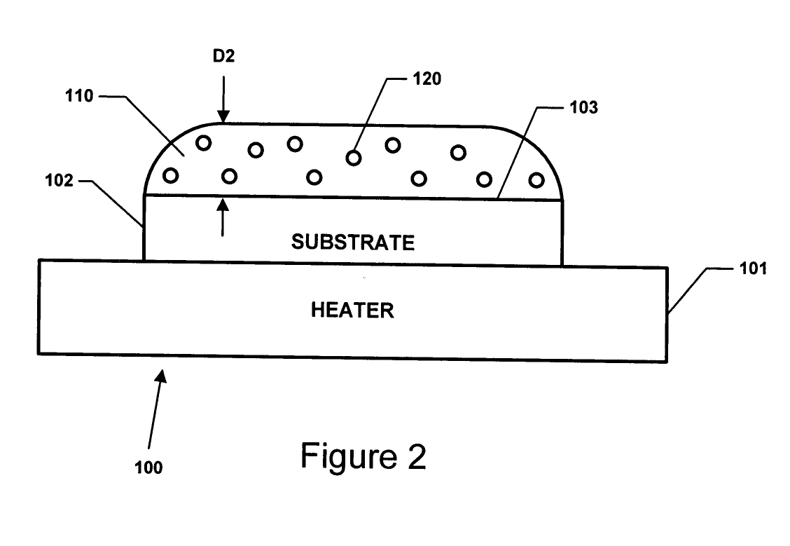

Referring now to FIG. 2, the sample 110 includes particles 120 suspended in the sample 110. The particles 120 can be added to the sample 110 either before or after adding the sample 110 to the substrate 102 or the chamber 105 of the container 104. The particles are distributed throughout substantially the entire volume of the sample.

Particles 120 within sample 110 are constantly moving. The particle motion can be induced by different phenomena or mechanisms such as Brownian motion, an external force such as a temperature gradient within sample 110, or agitating. When particles 120 move within the sample 110, they repel the front surrounding sample 110, which results in an empty wake in a rear region After a sufficient amount of time (at order of second for example), the surrounding sample 110 flows back to fill the empty wake region. Because the particle movement is typically of a random-walk nature (i.e., non-unidirectional movement) and mostly chaotic, it produces localized mixing in its surrounding region in a dimension of about 1 to about 100 times the particle dimension.

In general, particles 120 increase the volume of the sample 110. In some embodiments, increasing the volume of the sample 110 increases the depth D1 of the sample to a depth D2. In one embodiment, the depth D2 of the sample 110 having particles is about 10 to about 200 percent greater than the depth D1 of the sample 110 not having particles, although other ranges are possible. The increased depth provides a reduced complexity of the sample.

The quantity of the particles 120 included in the sample is chosen on the basis of the particle characteristics (e.g., size, surface, shape, wetability, porosity), compatibility with the sample, volume of the sample, compatibility with sample container including reacting substrate, suspension/dispersion, and other characteristics. In various possible embodiments, for example, the quantity of the particles 120 is from about 0.001% to about 100% weight over sample volume (w/v), from about 0.001% to about 10% (w/v), and from about 0.001% to about 1% (w/v), although other quantities are possible.

In some example embodiments, the particles 120 are generally spherical. However, the shape of the particles 120 is not so limited and particles 120 having any shape, including an irregular shape, may be used. In general, the particles 120 range in diameter from about 10 nm to about 10 microns. In one example embodiment, the particles 120 are about 5 microns in diameter, although other dimensions are possible. Particles 120 of these dimensions are sized to cause local movement within a sample without disrupting the target spots 103 on the substrate 102.

In some embodiments, the particles 120 are polyballs formed from one or more polymers, although other types of particles can be used. Example materials used to form the polyball particles 120 includes polystyrene, polyethylene, polypropylene, silicone, and various other copolymers. However, other suitable materials known to one with the relevant skill in the art may also be used. In general, the material used to form the particles 120 is chosen based on the desired surface properties of the particles 120.

In some embodiments, the particles 120 are non-porous on their surface and body. The specific surface area of the particles is very small, for example, less than about 10 m²/gram. When non-porous particles 120 are used, they increase the sample volume 110 by the aggregate volume of particles 120. Additionally, non-porosity reduces the surface adsorption of sample analyte onto the particle surface, reliving the analyte concentration requirement for reaction.

In general, the material used to form the particles 120 is chosen to prevent or reduce the likelihood of reaction with the sample 110 or substrate 102. In some example embodiments, the material used to form the particles 120 is chosen based on the wettibility of the surface to the sample 110 or the substrate 102. In other example embodiments, the material used to form the particles 120 is chosen based on the likelihood of the material to absorb the sample 110 or otherwise react with the sample 110.

Referring to FIG. 3, mixing the sample 110 enables the analytes within the sample 110 to spread out over the volume of the sample, thereby increasing the probability of each analyte contacting the target spots 103 on the substrate 102 configured to react with that analyte.

In some embodiments, the sample 110 is mixed, at least locally, by inducing random-walk movement of the particles 120 within the sample 110. Generally, the particles 120 are already in motion within the sample 110 to a degree due to a phenomenon called Brownian motion. The speed and displacement of the Brownian movement are generally dependent on the sample temperature that is controlled by the heater 101.

For example, in use, the heater 101 radiates thermal energy that flows to the sample 110. The thermal energy of the sample is then passed to the particles 120 suspended in the sample 110. In some embodiments, heating the particles 120 raises the temperature and, hence, the thermal energy of the particles 120. The increase in thermal energy causes an increase in the Brownian motion of the particles 120, which causes the particles 120 to move more quickly and violently, which, in turn, causes localized turbulence that mixes the analytes within the sample 110. While the turbulence is localized around each particle, the particles are distributed through the sample 110. This distribution of particles 120 provides simultaneous and efficient mixing of the entire sample 110.

In addition, in other embodiments, the heated particles 120 impact upon the molecules of the sample 110, thereby inducing movement of the sample molecules. Likewise, in still other embodiments, the heated particles 120 impact upon the analyte within the sample, thereby inducing movement of the analyte. Movement of the sample molecules and movement of the analyte both cause further turbulence within the sample 110.

In still other embodiments, heating the sample 110 causes the sample 110 to evaporate. Evaporation of the sample 110 can induce movement of the particles 120 suspended in the sample 110 and movement of the molecules composing the sample 110.

In yet still other embodiments, the heater 101 is configured to heat the substrate 102 to produce a temperature gradient, which is a gradual change in temperature, along the depth D of the sample 110. In some embodiments, however, cooling the substrate 102 may also produce a temperature gradient. This change in temperature further increases thermally induced movement of the particles 120 in addition to movement caused by the Brownian motion.

In one embodiment, the heater 101 heats the substrate 102 to produce a gradient of about 10 degrees along the depth D of the sample 110. In another embodiment, the heater 101 heats the substrate 102 to produce a gradient of about 1 degree along the depth D of the sample 110. In still another embodiment, the heater 101 heats the substrate 102 to produce a gradient of about 0.1 degree along the depth D of the sample 110. In other embodiments, however, gradients of other temperature ranges are also possible. Of course, a temperature gradient may also be created along the length L or a width (not shown) of the sample 110.

In general, movement of the heated particles 120 by about 1 nm to about 10 nm is sufficient to induce enough turbulence within the sample 110 to mix the sample 110 adequately. However, in other embodiments, greater movement, for example about 5 microns to about 10 microns, may also be suitable. However, the movement of the particles 120 should not be sufficient to interfere with the bond between the target spots 103 and the substrate 102.

In some embodiments, the apparatus 100 further includes a thermal controller 115 to operate the heater 101. The thermal controller 115 is configured to enable a user to select a desired temperature of the heater 101. In one embodiment, the thermal controller 115 is further configured to enable a user to select a period of time during which the heater 101 will operate.

In another exemplary embodiment, referring to FIG. 4, the substrate can be positioned within a container. The apparatus 200 includes a container 104 having a chamber 105 configured to house a substrate 102′. The sample 110′ containing at least one analyte can be added to the chamber 105.

Generally, the sample 110′ is inserted with sufficient quantity to immerse the substrate 102′. Surface tension is not necessary for retaining the sample 110′ in contact with the substrate 102′. In some example embodiments, the depth D1′ of sample 110′ ranges from about 0.01 mm to about 10 mm. However, in other embodiments, any suitable quantity of sample 110′ can be used.

In the illustrated embodiment shown in FIG. 4, the container 104 is positioned on the heater 101 to heat the container 104 and, thereby, the substrate 102′ and sample 110′. In some embodiments, the container 104 defines at least one chamber 105 configured to hold the substrate 102′ and the sample 110′. In one example embodiment, the chamber 105 is sealed from the surroundings of the container 104.

In some embodiments, the chamber 105 is dimensioned to hold a low volume of the sample 110′. The chamber 105 includes a length L′ and a depth H. In some example embodiments, the length L′ of the chamber 105 ranges from about 1 mm to about 100 mm and the depth H of the chamber 105 ranges from about 0.01 mm to about 10 mm, although other dimensions are possible. In one example embodiment, the length L′ of the chamber 105 is substantially greater than the depth H. In a particular example, embodiment, the ratio L′/H is greater than 10, in another example the ratio is greater than 100, in yet another example, the ration is greater than 1,000. However, in other embodiments, the chamber 105 can be any desired dimension.

In some embodiments, the container 104 also includes a cover 108. In one embodiment, the cover 108 is monolithically formed with the container 104. In other embodiments, the cover 108 is releasably coupled to the container 104 and enables insertion and removal of the sample 110′ from the container 104. In one such embodiment, the cover 108 is coupled to the container 104 via seal member 109.

In some embodiments, the substrate 102′ contained within the chamber 105 includes at least one microarray. In one embodiment, an active surface of the microarray, to which features 103′ are bonded, is positioned to face a side of the container 104 opposite the cover 108. In another embodiment, the microarray is positioned to face the cover 108 of the container 104.

In general, the chamber 105 has a height H that is greater than a depth D1′ of the sample 110′. In some embodiments, the chamber 105 is further configured to contain a gas layer intermediate the sample 110′ and the cover 108. However, in other embodiments, the sample 110′ may fill the chamber 105 entirely.

In some embodiments, the container 104 further includes an inlet 106 and outlet 107. The inlet 106 is arranged and configured to enable insertion of the sample 110′ into the chamber 105 of the container 104. The outlet 107 is arranged and configured to enable emptying of the sample 110′ from the chamber 105 of the container 104. In other embodiments, the inlet 106 and outlet 107 enable insertion and emptying of the gas layer into the chamber 105.

Referring now to FIG. 5, in some embodiments, particles 120′ can be added to the sample 110′ after the sample 110′ is injected into the container 104. In other embodiments, the particles 120′ are already suspended in the sample 110′ when the sample 110′ is added to the container 104. FIG. 5 illustrates movement of the particles 120′ within the sample 110′ contained in apparatus 200.

In general, the particles 120′ increase the depth of the sample 110′ from a depth D1′ to a depth D2′. In some embodiments, the depth D2′ is between 10 and 200 percent greater than the depth D1′. In other embodiments, however, other ranges are possible.

In general, heating the container 104 with the heater 101 operates the same as heating the substrate 102 directly as discussed above with respect to FIG. 3. In use, turbulence is induced within the sample 110 by at least one of heating the particles 120′, creating a thermal gradient within the sample 110′, and evaporating the sample 110′.

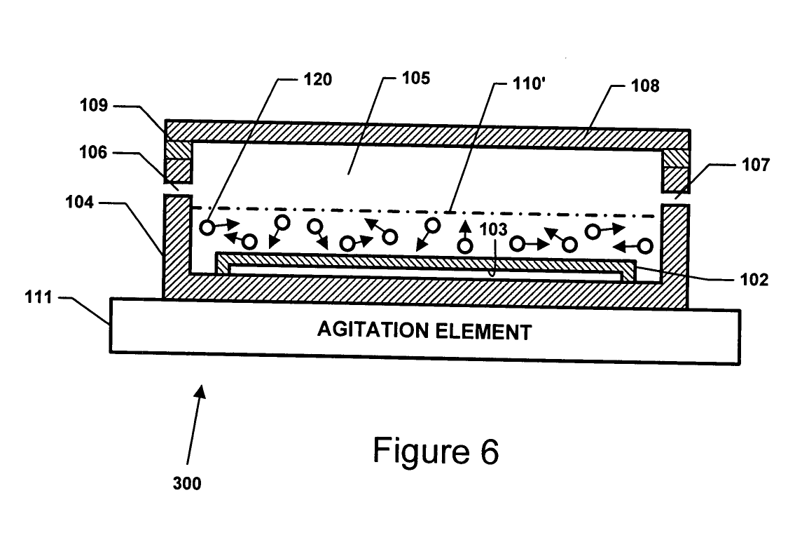

Referring now to FIG. 6, an agitation element can be used in place of the heater to induce movement of the particles within the sample. Particles tend to move within a suspension medium (e.g., the sample 110″) under acceleration/deceleration (Stokes flow). FIG. 6 illustrates an exemplary embodiment of an apparatus 300 for localized mixing of a low-volume sample 110″ containing at least one analyte. The apparatus 300 includes a substrate 102″, a container 104′, and an agitation element 111.

In some embodiments, the agitation element 111 includes a device configured to vibrate the container 104 and, hence, the particles 120′ suspended in the sample 110′. In other embodiments, the agitation element 111 is configured to oscillate the container 104. In still other embodiments, the agitation element 111 is configured to apply ultrasonic waves to the sample 110′ to induce movement of the particles 120′ within the sample 110′. In yet still other embodiments, the agitation element 111 is configured to stir the particles 120′ suspended in the sample 110′.

Additionally, the particles 120 and substrate 102 can be packaged in a kit. For example, a kit could include a substrate and a package containing a plurality of particles 120. The package of particles 120 can be added to a sample that is exposed to the substrate 102. In various embodiments, the package of particle 120 can contain a predetermined amount of particles for a predetermined volume of sample. In another example, the kit can include packages of particles 120 having different volumes of particles 120, each volume of particles 120 corresponding to a different volume of sample. In another example, a package of particles 120 can include a volume of particles 120 large enough for more than one sample. A kit could also contain packages of two or more different kinds of particles 120. A kit can also include a plurality of substrates. In yet another embodiment, the kit contains one or more containers 104 to receive the array 102, particles 120, and sample.

Arrays processed using the methods and structures disclosed herein find use in a variety of different applications, where such applications are generally analyte detection applications in which the presence of a particular analyte (i.e., target) in a given sample is detected at least qualitatively, if not quantitatively. Protocols for carrying out such assays are well known to those of skill in the art and need not be described in great detail here. Generally, the sample suspected of containing the analyte of interest is contacted with an array according to the subject methods and structures under conditions sufficient for the analyte to bind to its respective binding pair member (i.e., probe) that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g. an isotopic or fluorescent label present on the analyte, etc. The presence of the analyte in the sample is then deduced from the detection of binding complexes on the substrate surface. Specific analyte detection applications of interest include, but are not limited to, hybridization assays in which nucleic acid arrays are employed.

In these assays, a sample to be contacted with an array may first be prepared, where preparation may include labeling of the targets with a detectable label, e.g. a member of signal producing system. Generally, such detectable labels include, but are not limited to, radioactive isotopes, fluorescers, chemiluminescers, enzymes, enzyme substrates, enzyme cofactors, enzyme inhibitors, dyes, metal ions, metal sols, ligands (e.g., biotin or haptens) and the like. Thus, at some time prior to the detection step, described below, any target analyte present in the initial sample contacted with the array may be labeled with a detectable label. Labeling can occur either prior to or following contact with the array. In other words, the analyte, e.g., nucleic acids, present in the fluid sample contacted with the array according to the subject methods and structures may be labeled prior to or after contact, e.g., hybridization, with the array. In some embodiments of the subject methods, the sample analytes e.g., nucleic acids, are directly labeled with a detectable label, wherein the label may be covalently or non-covalently attached to the nucleic acids of the sample. For example, in the case of nucleic acids, the nucleic acids, including the target nucleotide sequence, may be labeled with biotin, exposed to hybridization conditions, wherein the labeled target nucleotide sequence binds to an avidin-label or an avidin-generating species. In an alternative embodiment, the target analyte such as the target nucleotide sequence is indirectly labeled with a detectable label, wherein the label may be covalently or non-covalently attached to the target nucleotide sequence. For example, the label may be non-covalently attached to a linker group, which in turn is (i) covalently attached to the target nucleotide sequence, or (ii) comprises a sequence which is complementary to the target nucleotide sequence. In another example, the probes may be extended, after hybridization, using chain-extension technology or sandwich-assay technology to generate a detectable signal (see, e.g., U.S. Pat. No. 5,200,314).

In certain embodiments, the label is a fluorescent compound, i.e., capable of emitting radiation (visible or invisible) upon stimulation by radiation of a wavelength different from that of the emitted radiation, or through other manners of excitation, e.g. chemical or non-radiative energy transfer. The label may be a fluorescent dye. Usually, a target with a fluorescent label includes a fluorescent group covalently attached to a nucleic acid molecule capable of binding specifically to the complementary probe nucleotide sequence.

Following sample preparation (labeling, pre-amplification, etc.), the sample may be introduced to the array. The sample is contacted with the array under appropriate conditions using the subject methods and structures to form binding complexes on the surface of the substrate by the interaction of the surface-bound probe molecule and the complementary target molecule in the sample. The presence of target/probe complexes, e.g., hybridized complexes, may then be detected. In the case of hybridization assays, the sample is typically contacted with an array under stringent hybridization conditions, whereby complexes are formed between target nucleic acids that agent are complementary to probe sequences attached to the array surface, i.e., duplex nucleic acids are formed on the surface of the substrate by the interaction of the probe nucleic acid and its complement target nucleic acid present in the sample. A “stringent hybridization” and “stringent hybridization wash conditions” in the context of nucleic acid hybridization (e.g., as in array, Southern or Northern hybridizations) are sequence dependent, and are different under different experimental parameters.

The array is then incubated with the sample under appropriate array assay conditions, e.g., hybridization conditions, as mentioned above, where conditions may vary depending on the particular biopolymeric array and binding pair. Once incubation is complete, the array is typically washed at least one time to remove any unbound and non-specifically bound sample from the substrate; generally at least two wash cycles are used. Washing agents used in array assays are known in the art and, of course, may vary depending on the particular binding pair used in the particular assay. For example, in those embodiments employing nucleic acid hybridization, washing agents of interest include, but are not limited to, salt solutions such as sodium, sodium phosphate (SSP) and sodium, sodium chloride (SSC) and the like as is known in the art, at different concentrations and which may include some surfactant as well.

Following the washing procedure, the array may then be interrogated or read to detect any resultant surface bound binding pair or target/probe complexes, e.g., duplex nucleic acids, to obtain signal data related to the presence of the surface bound binding complexes, i.e., the label is detected using colorimetric, fluorimetric, chemiluminescent, bioluminescent means or other appropriate means. The obtained signal data from the reading may be in any convenient form, i.e., may be in raw form or may be in a processed form.

In using an array processed using the subject methods and structures set forth herein, the array typically is exposed to a sample (for example, a fluorescently labeled analyte, e.g., protein containing sample) and the array then read. Reading of the array to obtain signal data may be accomplished by illuminating the array and reading the location and intensity of resulting fluorescence (if such methodology was employed) at each feature of the array to obtain a result. For example, an array scanner may be used for this purpose that is similar to the Agilent MICROARRAY SCANNER available from Agilent Technologies, Palo Alto, Calif. Other suitable apparatus and methods for reading an array to obtain signal data are described in U.S. Pat. Nos. 6,756,202 and 6,406,849, the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583, the disclosure of which is herein incorporated by reference, and elsewhere).

As noted above, the arrays processed according to the subject method and structure may be employed in a variety of array assays including hybridization assays. Specific hybridization assays of interest which may be practiced using the subject arrays include: gene discovery assays, differential gene expression analysis assays; nucleic acid sequencing assays, and the like. Patents describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

1. A method for low volume mixing, the method comprising:
providing a fluid having suspended particles and an analyte; and
heating the fluids thereby increasing movement of the suspended particles, wherein the movement of the suspended particles mixes the analyte within the fluid.

2. The method of claim 1, further comprising:
filling a container with the fluid through an inlet in the container, the container having a length and a depth, wherein the length of the container is substantially greater than the depth.

3. The method of claim 2, further comprising:
creating a thermal gradient along the depth of the container, the thermal gradient having a range of about one degree.

4. The method of claim 3, further comprising creating a thermal gradient within the fluid, the thermal gradient having a range of about one to about ten degrees.

5. The method of claim 1, wherein altering a temperature of the solution includes heating the fluid, and wherein heating the fluid induces Brownian motion of the particles.

6. The method of claim 1, further comprising evaporating a portion of the fluid, wherein evaporation of the solution induces movement of the suspended particles.

7. The method of claim 1, further comprising stirring the fluid including the suspended particles to induce movement of the suspended particles.

8. The method of claim 1, further comprising providing a container configured to retain the fluid and oscillating the container to induce movement of the suspended particles in the fluid.

9. The system of claim 1, wherein suspending the particles in the fluid comprises increasing a volume of the fluid by an amount ranging from about 1 percent to about 200 percent.

10. The system of claim 1, wherein suspending the particles in the fluid comprises suspending particles having a diameter ranging from about 1 nanometer to about 10 microns.

11. The system of claim 1, wherein suspending the particles in the fluid comprises suspending particles selected from the group consisting of: polystyrene beads, polyethylene beads, and silicon beads, silica, glass, and combinations thereof.

12. A system for low volume mixing, the system comprising:
a substrate, the substrate configured to hold a fluid having an analyte and suspended particles; and
a heating element, the heating element configured to heat the substrate, wherein heating the substrate heats the particles suspended within the fluid causing the suspended particles to mix the analyte within the fluid.

13. The system of claim 12, wherein the heating element raises the temperature of the substrate by about 10 degrees.

14. The system of claim 12, wherein the substrate comprises a microarray.

15. A system for low volume mixing, the system comprising:
a container, the container configured to retainably hold a fluid having an analyte and particles suspended in the fluid; and
an agitation element, the agitation element configured to impart movement to the container, wherein imparting movement to the container causes the suspended particles to move within the solution, thereby mixing the analyte within the solution.

16. The system of claim 16, further comprising a microarray positioned within the container, the microarray configured to contact the fluid.

17. The system of claim 16, further comprising a seal element, the seal element configured to couple to the container to seal the fluid within the container.

18. The system of claim 16, wherein the plurality of particles increase a volume of the analyte solution by an amount ranging from about 1 percent to about 200 percent.

19. The system of claim 16, wherein the heating element raises the temperature of the substrate by about 10 degrees.

20. A kit for low volume mixing, the kit comprising:
a microarray having a surface; and
a plurality of polymer particles, the polymer particles having a Browning motion when exposed to heat, wherein the polymer particles increase movement of an analyte suspended in a sample and across the surface of the microarray upon exposure of the polymer particles to a predetermined level of heat.

21. The kit of claim 20, further comprising a container sized to receive the microarray, the plurality of polymer particles, and a fluid.
